(19) United States
(12) Patent Application Publication
(10) Pub. No.: US 2015/0241428 A1
(43) Pub. Date: Aug. 27, 2015

(54) POINT-OF-CARE ASSAY FOR THE DETECTION OF ENTEROVIRUSES USING LATERAL FLOW IMMUNOCHROMATOGRAPHIC TECHNOLOGY

(71) Applicant: Innomart PTE LTD, Singapore (SG)

(72) Inventors: Jia Jun Lee, Singapore (SG); Eng Lee Tan, Singapore (SG)

(21) Appl. No.: 14/427,880

(22) PCT Filed: Sep. 12, 2013
(86) PCT No.: PCT/SG2013/000398

§ 371 Date: Mar. 12, 2015

(30) Foreign Application Priority Data
Sep. 12, 2012 (SG) 201206800-3

(51) Int. Cl. G01N 33/569; G01N 33/531; G01N 33/543

(52) U.S. Cl. CPC G01N 33/56983; G01N 33/54386; G01N 33/531

(57) ABSTRACT

The present disclosure relates to a point-of-care lateral flow immunochromatographic assay for direct detection of enteroviruses. In particular, the present disclosure relates to a point-of-care lateral flow immunochromatographic assay for detection of the etiologic agents of Hand, Foot and Mouth Disease (HFMD), using antibodies specific for enteroviruses.

### CROSS REFERENCE TO RELATED APPLICATIONS

This application claims priority to and is a conversion of Singapore Patent Application No. 201206800-3, entitled “Point-Of-Care Assay For The Detection Of Enteroviruses Using Lateral Flow Immunochromatographic Technology” filed on 12 Sep. 2012, which is incorporated herein by reference in its entirety.

### TECHNICAL FIELD

The present disclosure relates to a point-of-care lateral flow immunographic assay for detection of viruses. In particular, the present disclosure relates to a point-of-care lateral flow immunographic assay for detection of enteroviruses.

### BACKGROUND

Hand, Foot and Mouth Disease (HFMD) is one of the most common infectious diseases in the Asia Pacific region and has resulted in many fatalities over the last few decades. The disease is spread mainly by contact with body fluids, such as, saliva. Most enteroviruses are causative agents for HFMD. The prognosis for patients having HFMD can vary depending on the enterovirus causing the HFMD. For example, Enteroviruses, such as, Coxsackievirus A16 (CA16) typically cause self-limiting HFMD whereas Enterovirus 71 (EV71) can cause neurological complications and fatality.

HFMD is a form of mild exanthem that mainly affects children and results in vesicular rashes on hands, feet and/or the buccal mucosa. There are more than 60 enteroviruses that can cause HFMD. The main etiological agents of HFMD are enteroviruses belonging to the Picornaviridae family, namely EV71, CA16, Coxsackievirus B2 (CB2) and Echovirus 7 (Echo 7). In general, the clinical symptoms caused by most enteroviruses can be similar; thus, it can be difficult to determine which enterovirus has caused the HFMD by simply observing the clinical symptoms alone. However, EV71 is typically distinguishable due to its exceptional virulence, which can result in neurological complications and fatality. The neurological complications that can be caused by EV71 include aseptic meningitis, brainstem encephalitis, pulmonary edema and poliomyelitis-like paralysis.

Recent major outbreaks of HFMD in the Asia Pacific region resulted in 78 deaths in Taiwan in 1998, 41 deaths in Malaysia in 1997, 5 deaths in Singapore in 2000, 18 deaths in Malaysia in 2006, and 2 deaths in Singapore in 2008. All of the above fatalities were caused by EV71 and in some of these fatalities the patients were found to be co-infected with EV71 and CA16. In a recent HFMD epidemic in China in 2010, more than 250,000 HFMD cases including more than 250 fatalities due to EV71 were reported.

In Singapore in 2010 more than 19,000 HFMD cases were reported wherein 12% of the cases were caused by EV71. The alarming number of HFMD cases resulted in closures of childcare centres and schools, which eventually affected the economy.

To date, there are no effective antiviral drugs available for treating HFMD and there are no vaccines available for preventing HFMD. Presently, all known treatments for HFMD are symptomatic-based and have minimal effectiveness especially against EV71. With no effective antiviral drugs or vaccines currently available, there is a need for rapid and accurate identification of HFMD patients to provide timely treatment and management of patients and to reduce the spread of infection.

Further, currently, general practitioners, healthcare professionals, and school teachers typically classify, identify and/or diagnose children with HFMD or an early stage of HFMD based on visually observable symptoms, such as, a fever, mouth ulcers, and/or rashes on the extremities, such as, hands and/or feet. This method of diagnosis is very subjective and prone to misclassification or misdiagnosis. The spread of HFMD among children can be rapid especially in crowded places such as schools; thus, rapid and accurate identification of HFMD patients is needed to prevent the further spread of the disease.

### SUMMARY

The present disclosure relates to a lateral flow immunochromatographic assay that employs a specific antigen-antibody interaction. An analyte(s) containing a virus or viruses is added to a sample pad and subsequently flows along a test strip by capillary action. The test strip includes detector molecules, wherein the detector molecules comprise a first specific antibody conjugated to colloidal gold (i.e., colloidal gold nanoparticles). When the virus encounters the detector molecules, the first specific antibody that is conjugated to the colloidal gold recognises viral antigens of the virus, wherein the viral antigens comprise specific viral surface proteins (i.e., VP1 proteins of enteroviruses). The first specific antibody binds to a first region of the specific viral surface proteins (i.e., VP1 proteins of enteroviruses) of the virus to form a first complex comprising an antibody-antigen complex. The antibody-antigen complex migrates further down to a test line comprising a second specific antibody coated on the test line, wherein the second specific antibody recognizes the specific viral surface proteins (i.e., VP1 proteins of enteroviruses). The second specific antibody binds to a second region of the specific viral surface proteins (i.e., VP1 proteins of enteroviruses). The virus binds to the second specific antibody coated on the test line. The binding of the second specific antibody to the virus forms a second complex comprising an antibody-antigen sandwich complex. The formation of one or more antibody-antigen sandwich complexes results in the formation of a red colour line that can be visible or visually observed due to the colloidal gold of the detector molecules as the colloidal gold nanoparticles have a red colour.

The lateral flow immunochromatograhic assay of the present disclosure and the method of using thereof to detect enteroviruses allows for rapid point-of-care identification of HFMD patients in 30 minutes or less with an accuracy of 90% or greater. The lateral flow immunochromatograhic assay of the present disclosure and the method of using thereof to detect enteroviruses allows for effective and efficient patient management to be carried out and reduces any possibility of misdiagnosis.

A first aspect of the present disclosure provides a lateral flow test strip for detecting the presence of an enterovirus, wherein the test strip can comprise: a conjugate pad comprising a plurality of detector molecules; and a test line comprising a plurality of capture molecules; wherein the test strip detects an enterovirus.

In some embodiments, the lateral flow test strip described above can be specific for detecting enteroviruses. In some embodiments, the lateral flow test strip described above can be 100% specific for detecting enteroviruses.

In some embodiments, each of the detector molecules can comprise a first antigen-specific antibody conjugated to a detectable particle. In some embodiments, the detectable particle can comprise a gold nanoparticle. In some embodiments, the first antigen-specific antibody can be specific for a viral antigen comprising a viral surface protein. In some embodiments, the first antigen-specific antibody can be specific for a first region of the viral surface protein. In some embodiments, the viral surface protein can be a VP1 protein. In some embodiments, each of the detector molecules can comprise one or more first antigen-specific antibodies, wherein each of the one or more first antigen-specific antibodies can be conjugated to a detectable particle.

In some embodiments, each of the capture molecules comprises a second antigen-specific antibody. In some embodiments, the second antigen-specific antibody can be specific for the viral surface protein. In some embodiments, the second antigen-specific antibody can be specific for a second region of the viral surface protein. In some embodiments, the viral surface protein can be a VP1 protein.

In some embodiments, the lateral flow test strip described above can provide test results in about 30 minutes or less, about 20 minutes or less or about 10 minutes. In some embodiments, the lateral flow test strip can provide test results in about 20 minutes or less. In some embodiments, the lateral flow test strip described above can provide test results in about 10 minutes.

In some embodiments, the lateral flow test strip described above can provide test results with an accuracy of about 90% or greater. In some embodiments, the lateral flow test strip described above can provide test results with an accuracy of about 95% or greater. In some embodiments, the lateral flow test strip described above can provide test results with an accuracy of about 99% or greater. In some embodiments, the lateral flow test strip described above can provide test results with an accuracy of about 100%.

In some embodiments, the enterovirus can comprise one or more enteroviruses.

In some embodiments, the lateral flow test strip described above can be a point-of-care lateral flow test strip.

A second aspect of the present disclosure provides a method of manufacturing the lateral flow test strip described above, wherein the method can comprise: making a conjugate pad; preparing a detector molecule solution comprising detector molecules; and soaking the conjugate pad in the detector molecule solution.

A third aspect of the present disclosure provides a diagnostic kit for detecting one or more enteroviruses, wherein the diagnostic kit comprises the lateral flow test strip described above.

In some embodiments, the diagnostic kit described above further comprises a sample collection device for collecting a biological fluid sample.

A fourth aspect of the present disclosure provides a method of determining the presence of an enterovirus using a lateral flow immunochromatographic assay, wherein the method can comprise: providing a sample; providing a lateral flow test strip comprising a plurality of detector molecules and a plurality of capture molecules; adding the sample to a sample pad of the lateral flow test strip; and exposing the sample to the plurality of detector molecules.

In some embodiments, each of the detector molecules can comprise a first antigen-specific antibody conjugated to a detectable particle. In some embodiments, the detectable particle can comprise a gold nanoparticle. In some embodiments, each of the detector molecules can comprise one or more first antigen-specific antibodies, wherein each of the one or more first antigen-specific antibodies can be conjugated to a detectable particle

In some embodiments, the first antigen-specific antibody can be specific for a viral antigen comprising a viral surface protein, wherein if the sample comprises an enterovirus the first antigen-specific antibody can bind to the viral surface protein to form one or more first complexes. In some embodiments, the first antigen-specific antibody can be specific for a first region of the viral surface protein. In some embodiments, the viral surface protein can be a VP1 protein.

In some embodiments, each of the capture molecules can comprise a second antigen-specific antibody. In some embodiments, the one or more first complexes can be exposed to the second antigen-specific antibody, wherein the second antigen-specific antibody can be specific for the viral surface protein, wherein the second antigen-specific antibody can bind to the viral surface protein to form one or more second complexes. In some embodiments, the second antigen-specific antibody can be specific for a second region of the viral surface protein. In some embodiments, the viral surface protein can be a VP1 protein.

In some embodiments, the formation of the one or more second complexes can result in the formation of a red colour line, wherein the red colour line indicates the presence of an enterovirus.

In some embodiments, the sample can comprise a biological fluid. In some embodiments, the biological fluid can comprise saliva.

In some embodiments, the enterovirus can comprise-one or more enteroviruses.

In some embodiments, the lateral flow immunochromatographic assay can be a point-of-care lateral flow immunochromatographic assay.

In some embodiments, the method of determining the presence of an enterovirus described above can provide test results in about 30 minutes or less. In some embodiments, the method of determining the presence of an enterovirus described above can provide test results in about 20 minutes or less. In some embodiments, the method of determining the presence of an enterovirus described above can provide test results in about 10 minutes.

In some embodiments, the method of determining the presence of an enterovirus described above can provide test results with an accuracy of about 90% or greater. In some embodiments, the method of determining the presence of an enterovirus described above can provide test results with an accuracy of about 95% or greater. In some embodiments, the method of determining the presence of an enterovirus described above can provide test results with an accuracy of about 99% or greater. In some embodiments, the method of determining the presence of an enterovirus described above can provide test results with an accuracy of 100%.

### BRIEF DESCRIPTION OF DRAWINGS

FIG. 1 illustrates the principle, concepts and/or components of a lateral flow immunochromatographic assay in accordance with an embodiment of the present disclosure.

FIG. 2 illustrates a set-up of a lateral flow immunochromatographic test strip in accordance with an embodiment of the present disclosure.

FIG. 3 shows experimental results of test runs using an embodiment of a lateral flow immunochromatographic test strip of the present disclosure to detect different types of viruses, wherein C is the control line and T is the test line.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments can be utilized, and other changes can be made, without departing from the spirit or scope of the subject matter presented herein.

Unless specified otherwise, the terms “comprising” and “comprise” as used herein, and grammatical variants thereof, are intended to represent “open” or “inclusive” language such that they include recited elements but also permit inclusion of additional, un-recited elements.

As used herein, the term “about”, in the context of concentrations of components, conditions, other measurement values, etc., means+/−5% of the stated value, or +/−4% of the stated value, or +/−3% of the stated value, or +/−2% of the stated value, or +/−1% of the stated value, or +/−0.5% of the stated value, or +/−0% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The present disclosure relates to a lateral flow test strip for detecting the presence of an enterovirus, wherein the test strip can comprise: a conjugate pad; and a test line; wherein the conjugate pad comprises detector molecules; wherein the test strip detects an enterovirus.

In some embodiments, the lateral flow test strip can be specific for detecting enteroviruses. In some embodiments, the lateral flow test strip can be 100% specific for detecting enteroviruses.

In some embodiments, each of the detector molecules can comprise a first antigen-specific antibody conjugated to a detectable particle. In some embodiments, the detectable particle can comprise a gold nanoparticle. In some embodiments, the gold nanoparticles can have a size of 100 nm or less, 40 nm or less, or 20 nm or less. Other detectable particles are also contemplated. In some embodiments, each of the detector molecules can comprise one or more first antigen-specific antibodies, wherein each of the one or more first antigen-specific antibodies can be conjugated to a detectable particle

In some embodiments, the first antigen-specific antibody can be specific for a viral antigen comprising a viral surface protein. In some embodiments, the first antigen-specific antibody can be a pan-enterovirus antibody. The use of other antibodies is also contemplated.

In some embodiments, detector molecules can be prepared by conjugating protein A coated colloidal gold (i.e., protein A coated colloidal gold nanoparticles) to pan-enterovirus antibodies according to the following protocol:

1. 2 mL of Collodial gold solution comprising gold nanoparticles can be added into an eppendorf tube and spun at 4° C., 25000g for 1 hr.
2. The supernatant can be discarded. The pellet can be re-suspended with 1 mL of Protein A IgG Binding Buffer (ThermoScientific).
3. A sufficient volume of pan-enterovirus antibodies can be added to the resultant mixture of step (2) to prepare a solution having a final concentration of 0.1 to 0.3 mg/mL.
4. The resultant solution of step (3) can be incubated for 1 hour with gentle shaking on a thermomixer at 450 rpm at 25° C.
5. After incubation, the solution can be spun down at 4° C. at 25000g for 1 hr. The pellet can be resuspended in 0.5 to 1 mL of Protein A IgG Binding Buffer (ThermoScientific) to form a final solution comprising protein A coated gold nanoparticles conjugated to pan-enterovirus antibodies.
6. The final solution comprising protein A coated gold nanoparticles conjugated to pan-enterovirus antibodies can be stored at 4° C. until further use.

In some embodiments, the test line can comprise a plurality of capture molecules. In some embodiments, each capture molecule can comprise a second antigen-specific antibody that is coated on the test line. In some embodiments, the second antigen-specific antibody can be specific for the viral surface protein. In some embodiments, the second antigen-specific antibody can be an anti-enterovirus 71 antibody. The use of other antibodies is also contemplated.

In some embodiments, test line capture molecules can be prepared by conjugating biotin to anti-enterovirus 71 antibodies (Millipore, Mass., USA) according to the following protocol:

1. 50 mM EZ-Link® Amine-PEG2-Biotin (ThermoScientific, Massachusetts, USA) can be prepared in 0.1M MES buffer to form a Biotin-MES solution.
2. 0.5 to 1 mL of 0.1M MES buffer can be added to 50 uL to 100 uL (i.e., of the Biotin-MES solution.
3. A sufficient volume of anti-enterovirus 71 antibodies can then be added to the solution to prepare a MES-Biotin-Antibody solution having a concentration of 1.0 mg/mL to 2.0 mg/mL.
4. 1 to 5 uL of 10 mM EDC (1-Ethyl-3-(3-dimethylaminopropyl)carbodiimide) can be added into the MES-Biotin-Antibody solution.
5. The resultant solution can be incubated for 2 hrs with gentle shaking at room temperature.
6. After incubation the resultant solution can be centrifuged at 14000g for 10 mins and the biotin-antibody supernatant can be collected.
7. The biotin-antibody supernatant can then be subjected to a desalting process:
a. A desalting column can be opened and inserted into a 15 mL falcon tube before spinning it down once at 1000g for 2 mins.
c. The falcon tube can be discarded. The desalting column can be inserted into a new 15 mL falcon tube and overlaid with the biotin-antibody supernatant obtained from step (6) and spun at 1000g for 2 mins.
d. The biotin-antibody supernatant can be collected and the desalting column can be discarded.
e. The biotin-antibody supernatant collected can be stored at −20° C. until further use.

In some embodiments, control line capture molecules can be prepared by conjugating biotin to goat anti-mouse IgG antibodies (ThermoScientific) and ten streptavidin according to the following protocol:

1. 50 mM EZ-Link® Amine-PEG2-Biotin (ThermoScientific) can be prepared in 0.1M MES buffer to form a Biotin-MES solution.
2. 0.5 to 1 mL of 0.1M MES buffer can be added to 50 uL to 100 uL of the Biotin-MES solution.
3. A sufficient volume of goat anti-mouse IgG antibodies (ThermoScientific) can then be added to the solution to prepare an MES-Biotin-Antibody solution having a concentration of 2.0 mg/mL.
4. 1 to 5 uL of 10 mM EDC can be added into the MES-Biotin-Antibody solution.
5. The resultant solution can be incubated for 2 hrs with gentle shaking at room temperature.
6. After incubation, the resultant solution can be centrifuged at 14000g for 10 mins and the biotin-antibody supernatant can be collected.
7. The biotin-antibody supernatant can then be subjected to desalting:
a. A desalting column can be opened and inserted into a 15 mL falcon tube before spinning it down once at 1000g for 2 mins.
c. The falcon tube can be discarded. The desalting column can be inserted into a new 15 mL falcon tube and overlaid with biotin-antibody supernatant obtained from step (6) and spun at 1000g for 2 mins.
d. The biotin-antibody supernatant can be collected and the desalting column can be discarded.
e. The biotin-antibody supernatant collected can be stored at −20° C. until further use.
8. 100 to 500 μL of 1 mg/ml Streptavidin (Sigma-Aldrich, St. Louis, Mo., USA) can be added to the biotin-antibody supernatant solution and mixed thoroughly.
9. The final biotin-antibody solution can be stored at −20° C. until further use.

In some embodiments, the lateral flow test strip can provide test results in about 30 minutes or less, about 25 minutes or less, about 20 minutes or less, about 15 minutes or less, or about 10 minutes. For clinical samples, after 35 minutes all test results of the lateral flow test strip are positive regardless of whether or not an enterovirus is present in the clinical sample.

In some embodiments, the lateral flow test strip can provide test results with an accuracy of about 90% or greater, about 95% or greater, about 99% or greater or about 100%.

In some embodiments, the lateral flow test strip can detect the presence of two or more enteroviruses simultaneously.

In some embodiments, the lateral flow test strip can detect the presence of one or more enteroviruses In some embodiments, the lateral flow test strip of the present disclosure can detect the presence of all enteroviruses including echoviruses, coxsackie A and B viruses, polio 1, 2 and 3, and enteroviruses 70 and 71.

In some embodiments, the lateral flow test strip can be a point-of-care lateral flow test strip.

The present disclosure also relates to a method of manufacturing the lateral flow test strip of the present disclosure, wherein the method can comprise: making a conjugate pad; preparing a detector molecule solution comprising detector molecules; and soaking the conjugate pad in the detector molecule solution.

The present disclosure also relates to a diagnostic kit for detecting one or more enteroviruses, wherein the diagnostic kit comprises the lateral flow test strip of the present disclosure.

In some embodiments, the diagnostic kit further comprises a sample collection device for collecting a biological fluid sample or biological fluid samples. In some embodiments, the biological fluid sample can comprise saliva or urine. Other biological fluid samples are also contemplated.

The present disclosure also relates to a method of determining the presence of an enterovirus using a lateral flow immunochromatographic assay, wherein the method can comprise: providing a sample; providing a lateral flow test strip; adding the sample to a sample pad of the lateral flow test strip; and exposing the sample to detector molecules of the lateral flow test strip.

In some embodiments, each of the detector molecules can comprise a first antigen-specific antibody conjugated to a detectable particle. In some embodiments, the detectable particle can comprise a gold nanoparticle. In some embodiments, the gold nanoparticles can have a size of 100 nm or less, 40 nm or less, or 20 nm or less. Other detectable particles are also contemplated. In some embodiments, each of the detector molecules can comprise one or more first antigen-specific antibodies, wherein each of the one or more first antigen-specific antibodies can be conjugated to a detectable particle.

In some embodiments, the first antigen-specific antibody can be specific for a viral antigen comprising a viral surface protein, wherein if the sample comprises an enterovirus the antigen-specific antibody can bind to the viral surface protein to form one or more first complexes. In some embodiments, the first antigen-specific antibody can be a pan-enterovirus antibody. The use of other antibodies is also contemplated.

In some embodiments, each of the capture molecules comprises a second antigen-specific antibody. In some embodiments, the one or more first complexes can be exposed to the second antigen-specific antibody, wherein the second antigen-specific antibody can be specific for the viral surface protein, wherein the second antigen-specific antibody can bind to the viral surface protein to form one or more second complexes. In some embodiments, the second antigen-specific antibody can be an anti-enterovirus 71 antibody. The use of other antibodies is also contemplated.

In some embodiments, the formation of the one or more second complexes can result in the formation of a red colour line, wherein the red colour line indicates the presence of an enterovirus.

In some embodiments, the sample can comprise a biological fluid. In some embodiments, the biological fluid can comprise saliva or urine. Other biological fluids are also contemplated.

In some embodiments, the lateral flow test strip can detect the presence of two or more enteroviruses simultaneously.

In some embodiments, the lateral flow test strip can detect the presence of one or more enteroviruses In some embodiments, the lateral flow test strip of the present disclosure can detect the presence of all enteroviruses including echoviruses, coxsackie A and B viruses, polio 1, 2 and 3, and enteroviruses 70 and 71.

In some embodiments, the lateral flow immunochromatographic assay can be a point-of-care lateral flow immunochromatographic assay.

In some embodiments, the method of determining the presence of an enterovirus described above can provide test results in about 30 minutes or less, about 25 minutes or less, about 20 minutes or less, or about 10 minutes. After 35 minutes all test results of the lateral flow test strip are positive regardless of whether or not an enterovirus is present in the sample.

In some embodiments, the method of determining the presence of an enterovirus described above can provide test results with an accuracy of about 90% or greater, about 95% or greater, about 99% or greater, or about 100%.

The present disclosure relates to the use of antigen-specific antibodies conjugated to colloidal gold (i.e., colloidal gold nanoparticles) to detect enteroviruses that cause Hand, Foot and Mouth Disease (HFMD) using lateral flow immunochromatographic technology.

The present disclosure relates to a lateral flow immunochromatographic assay that employs a specific antigen-antibody interaction. An analyte(s) containing a virus or viruses is added to a sample pad and subsequently flows along a test strip by capillary action. The test strip includes detector molecules, wherein the detector molecules comprise a first specific antibody conjugated to colloidal gold (i.e., colloidal gold nanoparticles). When the virus encounters the detector molecules, the first specific antibody that is conjugated to the colloidal gold recognises viral antigens of the virus, wherein the viral antigens comprise specific viral surface proteins (i.e., VP1 proteins of enteroviruses). The first specific antibody binds to a first region of the specific viral surface proteins (i.e., VP1 proteins of enteroviruses) of the virus to form a first complex comprising an antibody-antigen complex. The antibody-antigen complex migrates further down to a test line comprising a second specific antibody coated on the test line, wherein the second specific antibody recognizes the specific viral surface proteins (i.e., VP1 proteins of enteroviruses). The second specific antibody binds to a second region of the specific viral surface proteins (i.e., VP1 proteins of enteroviruses). The virus binds to the second specific antibody coated on the test line. The binding of the second specific antibody to the virus forms a second complex comprising an antibody-antigen sandwich complex. The formation of one or more antibody-antigen sandwich complexes results in the formation of a red colour line that can be visible, or visually observed due to the colloidal gold of the detector molecules as the colloidal gold nanoparticles have a red colour.

The lateral flow immunochromatographic technology of the present disclosure uses antigen-antibody interaction that is very specific. The lateral flow, immunochromatographic technology of the present disclosure is user-friendly. As such, users or individuals that have no healthcare training can successfully use the lateral flow test strip and lateral flow immunochromatographic assay of the present disclosure. For example, the user only needs to apply a sample (i.e., a sample fluid) and a fixed amount of running buffer to the sample pad to initiate a test.

The lateral flow test strip of the present disclosure uses capillary action to test for enteroviruses. The lateral flow test strip of the present disclosure provides test results in 30 mins or less from the time a sample (i.e., a sample fluid) is applied to the sample pad.

The lateral flow test strip of the present disclosure can be manufactured such that the lateral flow test strip can be small and portable. In some embodiments, a lateral flow test strip can have a length of about 6 cm and width of about 0.5 cm. In some embodiments, the length can be greater or less than about 6 cm and the width can be greater or less than about 0.5 cm. Other dimensions for the lateral flow test strip are also contemplated. The test results are based on colour formation that is visually observable by the human eye; thus, the lateral flow immunochromatographic assay of the present disclosure does not require the use of an expensive optical detection instrument.

The lateral flow immunochromatograhic assay of the present disclosure and the method of using thereof to detect enteroviruses allows for rapid point-of-care identification of HFMD patients in 30 minutes or less with an accuracy of 90% or greater. The lateral flow immunochromatograhic assay of the present disclosure and the method of using thereof to detect enteroviruses allows for effective and efficient patient management to be carried out and reduces any possibility of misdiagnosis.

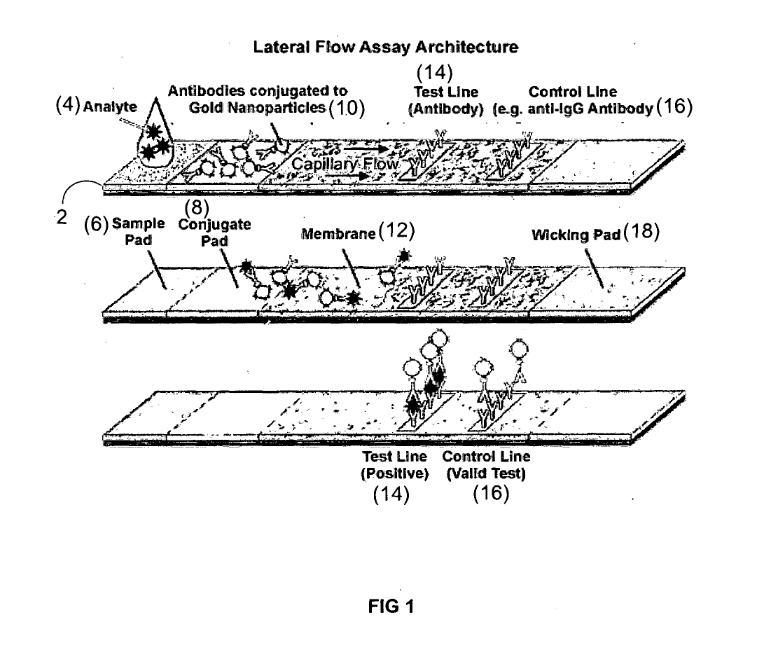

FIG. 1 illustrates the principle, concepts and/or components of a lateral flow immunochromatographic assay in accordance with an embodiment of the present disclosure.

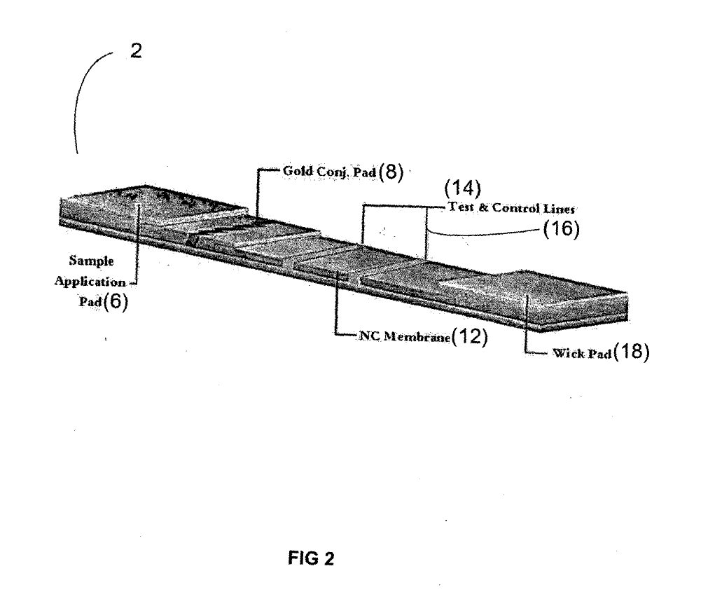

FIG. 2 illustrates a set-up of a lateral flow immunochromatographic test strip in accordance with an embodiment of the present disclosure.

As shown in FIG. 1 and FIG. 2, the testing and analysis of samples can be conducted by adding a sufficient volume of a biological fluid sample (i.e., saliva) comprising an analyte(s) 4 to a sample pad 6 of a lateral test strip 2, wherein the analyte(s) 4 can comprise one or more enteroviruses. When the sample pad 6 becomes saturated with the biological fluid sample comprising the analyte(s) 4, the biological fluid sample comprising the analyte(s) 4 can migrate to a conjugate pad 8 comprising detector molecules 10, wherein each of the detector molecules 10 comprises a first antigen-specific antibody conjugated to a gold nanoparticle, wherein the first antigen-specific antibody is specific to enteroviruses. The biological fluid sample comprising the analyte(s) 4 and the detector molecules 10 can mix flow through a capillary bed of the lateral flow test strip 2. In so doing, if the analyte(s) 4 contains an enterovirus, the first antigen-specific antibodies can bind to a first region of VP1 proteins of the enterovirus while migrating further through the capillary bed thereby forming one or more antibody-antigen complexes. If the analyte(s) 4 does not contain an enterovirus, the first antigen-specific antibodies do not bind to the viral surface proteins.

The lateral flow test strip 2 can comprise a membrane/reaction membrane 12. The membrane 12 can be a nitrocellulose membrane or cellulose acetate membrane that comprises a test line 14 that crosses the membrane 12. The test line 14 can comprise a second antigen-specific antibody that is immobilized on the test line 14.

The one or more antibody-antigen complexes can migrate further down to the test line 14, which is coated with the second antigen-specific antibody, wherein the second antigen-specific antibody is also specific for enteroviruses. The second antigen-specific antibodies can bind to a second region of the VP1 proteins of the enterovirus. The second antigen-specific antibodies can bind to the enterovirus thereby forming one or more antibody-antigen sandwich complexes. Over a period of time, as more of the biological fluid sample comprising the analyte(s) 4 has passed through the lateral flow test strip 2 more antibody-antigen sandwich complexes accumulate. As the antibody-antigen sandwich complexes accumulate the number of gold nanoparticles accumulates resulting in a visible or visually observable red colour line. The formation of a red colour line indicates a positive test confirming that the analyte(s) or biological fluid sample 4 contains an enterovirus. The red colour line can be visually observed by the human eye. The absence of a red colour line indicates a negative test confirming that the biological fluid sample does not contain an enterovirus.

The membrane 12 can also comprise a control line 16 that crosses the membrane 12. The control line 16 comprises a third antibody that is specific for the first antigen-specific antibody of the detector molecules 10.

The lateral flow test strip 2 can further comprise a wick pad/wicking pad 18. The wick pad 18 can be used to draw the biological fluid sample comprising the analyte(s) 4 across the membrane 12 by capillary action and collect the biological fluid sample.

### Example

### Experimental Set-Up

### 1. Buffer Preparation

### Sample Pad Buffer

10 mM Phosphate buffer, 1% v/v isopropanol and 0.04% Tween were added together to form a mixture. The pH of the mixture was adjusted to 8.0 thereby forming a sample pad buffer. The sample pad buffer was stored at 4° C.

### Conjugation Buffer

A conjugation buffer was prepared using 0.1M MES (2-(N-morpholino)ethanesulfonic acid) buffer having a pH adjusted to between 4.7-5.5. The conjugation buffer was stored at 4° C.

### Running Buffer

1 mM sodium borate, 1% of isopropanol and 0.04% of Tween were added together to form a mixture. The pH of the mixture was adjusted to 8.0 thereby forming a running buffer. The running buffer was stored at 4° C.

### 2. Lateral Flow Strip Construction

The construction of lateral flow test strips of the present disclosure is shown in FIG. 1. The assembly of lateral flow test strips was performed using a BioDot Batch Laminator System (BioDot, CA, USA) according to the manufacturer's instructions. Test line capture molecules and control line capture molecules were sprayed using a single coat onto a nitrocellulose membrane using a BioDot Dispensary Platform (BioDot) at a rate of 1 μl/cm. After coating the lateral flow test strip with the test line and control line, the nitrocellulose membrane was blocked by a blocking buffer (Life Technologies Corporation, CA, USA) for 20 mins with gentle shaking. A sample pad was soaked in the sample pad buffer and dried at 37° C. A gold conjugate pad was soaked in a detector molecule solution and dried at 37° C. The sample pad and gold conjugated pad were then added to the lateral flow test strip. All assembled test strips were stored in a dessicator until use.

### 3. Preparation of Test Line Capture Molecules

Test line capture molecules were prepared by conjugating biotin to anti-enterovirus 71 antibodies (Millipore, Mass., USA) according to the following protocol:
1. 50 mM EZ-Link® Amine-PEG₂-Biotin (ThermoScientific, Massachusetts, USA) was prepared in 0.1M MES buffer to form a Biotin-MES solution.
2. 1 mL of 0.1M MES buffer was added to 60 uL of the Biotin-MES solution.
3. A sufficient volume of anti-enterovirus 71 antibodies was then added to the solution to prepare a MES-Biotin-Antibody solution having a concentration of 2.0 mg/mL.
4. 3 uL of 10 mM EDC (1-Ethyl-3-(3-dimethylaminopropyl)carbodiimide) was added into the MES-Biotin-Antibody solution.
5. The resultant solution was incubated for 2 hrs with gentle shaking at room temperature.
6. After incubation the resultant solution was centrifuged at 14000g for 10 mins and the biotin-antibody supernatant was collected.
7. The biotin-antibody supernatant was then subjected to a desalting process:
   a. A desalting column was opened and inserted into a 15 mL falcon tube before spinning it down once at 1000g for 2 mins.
   c. The falcon tube was discarded. The desalting column was inserted into a new 15 mL falcon tube and overlaid with the biotin-antibody supernatant obtained from step (6) and spun at 1000g for 2 mins.
   d. The biotin-antibody supernatant was collected and the desalting column was discarded.
   e. The biotin-antibody supernatant collected was stored at −20° C. until further use.

### 4. Preparation of Detector Molecules

Detector molecules were prepared by conjugating protein A coated colloidal gold (i.e., protein A coated colloidal gold nanoparticles) to pan-enterovirus antibodies according to the following protocol:
1. 2 ml of Collodial gold solution comprising gold nanoparticles was added into an eppendorf tube and spun at 4° C., 25000g for 1 hr.
2. The supernatant was discarded. The pellet was re-suspended with 1 mL of Protein A IgG Binding Buffer (ThermoScientific).
3. A sufficient volume of pan-enterovirus antibodies was added to the resultant mixture of step (2) to prepare a solution having a final concentration of 0.3 mg/mL.
4. The resultant solution of step (3) was incubated for 1 hour with gentle shaking on a thermomixer at 450 rpm at 25° C.
5. After incubation, the solution was spun down at 4° C. at 25000g for 1 hr. The pellet was resuspended in 1 mL of Protein A IgG Binding Buffer (ThermoScientific) to form a final solution comprising protein A coated gold nanoparticles conjugated to pan-enterovirus antibodies.
6. The final solution comprising protein A coated gold nanoparticles conjugated to pan-enterovirus antibodies was stored at 4° C. until further use

### 5. Preparation of Control Line Capture Molecules

Control line capture molecules were prepared by conjugating biotin to goat anti-mouse IgG antibodies (ThermoScientific) and ten streptavidin according to the following protocol:
1. 50 mM EZ-Link® Amine-PEG₂-Biotin (ThermoScientific) was prepared in 0.1M MES buffer to form a Biotin-MES solution.
2. 1 mL of 0.1M MES buffer was added to 60 uL of the Biotin-MES solution.
3. A sufficient volume of goat anti-mouse IgG antibodies (ThermoScientific) was then added to the solution to prepare an MES-Biotin-Antibody solution having a concentration of 2.0 mg/mL.
4. 3 uL of 10 mM EDC was added into the MES-Biotin-Antibody solution.
5. The resultant solution was incubated for 2 hrs with gentle shaking at room temperature.
6. After incubation, the resultant solution was centrifuged at 14000g for 10 mins and the biotin-antibody supernatant was collected.
7. The biotin-antibody supernatant was then subjected to desalting:
   a. A desalting column was opened and inserted into a 15 mL falcon tube before spinning it down once at 1000g for 2 mins.
   c. The falcon tube was discarded. The desalting column was inserted into a new 15 mL falcon tube and overlaid with biotin-antibody supernatant obtained from step (6) and spun at 1000g for 2 mins.
   d. The biotin-antibody supernatant was collected and the desalting column was discarded.
   e. The biotin-antibody supernatant collected was stored at −20° C. until further use.
8. 250 μl of 1 mg/ml Streptavidin (Sigma-Aldrich, St. Louis, Mo., USA) was added to the biotin-antibody supernatant solution and mixed thoroughly.
9. The final biotin-antibody solution was stored at −20° C. until further use.

### Viruses Analysed

An embodiment of a lateral flow immunochromatographic assay and lateral flow test strip of the present disclosure was used to test for and analyse the following viruses, which were cultured in a laboratory:

### 1. Enteroviruses

Enterovirus 71 (EV71)
Coxsackievirus A16 (CA16)
Coxsackievirus B2 (CB2)
Echovirus 6 (Echo 6)

### 2. Non-Enteroviruses

Cytomegalovirus (CMV)
Herpes Simplex Virus (HSV)

The above-mentioned viruses were separately introduced into six (6) separate saliva fluid samples. As such, each of the six (6) saliva fluid samples comprised a different virus. Each of the six (6) saliva fluid samples were tested and analysed using a separate but identical lateral flow test strip of the present disclosure.

The testing and analysis was conducted by adding a sufficient volume of saliva fluid sample to the sample pad of the lateral test strip, wherein the saliva fluid sample comprises a virus. The volume of saliva fluid sample added can be from about 30 μl to about 100 μl. When the sample pad becomes saturated with the saliva fluid sample, the saliva fluid sample migrates to the gold conjugate pad comprising the detector molecules, wherein each of the detector molecules comprises a first antigen-specific antibody conjugated to a gold nanoparticle, wherein the first antigen-specific antibody is specific to enteroviruses. The saliva fluid sample and the detector molecules mix flow through a capillary bed of the lateral flow test strip. In so doing, if the saliva fluid sample contains an enterovirus, the first antigen-specific antibodies can bind to a first region of specific viral surface proteins (in this case VP1 proteins) of the enterovirus while migrating further through the capillary bed thereby forming one or more antibody-antigen complexes. If the saliva fluid sample does not contain an enterovirus, the first antigen-specific antibodies do not bind to the viral surface proteins.

The one or more antibody-antigen complexes migrate further down to the test line, which is coated with a second antigen-specific antibody, wherein the second antigen-specific antibody is also specific for enteroviruses. The second antigen-specific antibodies can bind to a second region of the specific viral surface proteins (in this case VP1 proteins) of the enterovirus thereby forming one or more antibody-antigen sandwich complexes. Over a period of time, as more of the saliva fluid sample has passed through the lateral flow test strip more antibody-antigen sandwich complexes accumulate. As the antibody-antigen sandwich complexes accumulate the number of gold nanoparticles accumulates resulting in a visible or visually observable red colour line. The formation of a red colour line indicates a positive test confirming that the saliva fluid sample contains an enterovirus. The red colour line can be visually observed by the human eye. The absence of a red colour line indicates a negative test confirming that the saliva fluid does not contain an enterovirus.

### Results

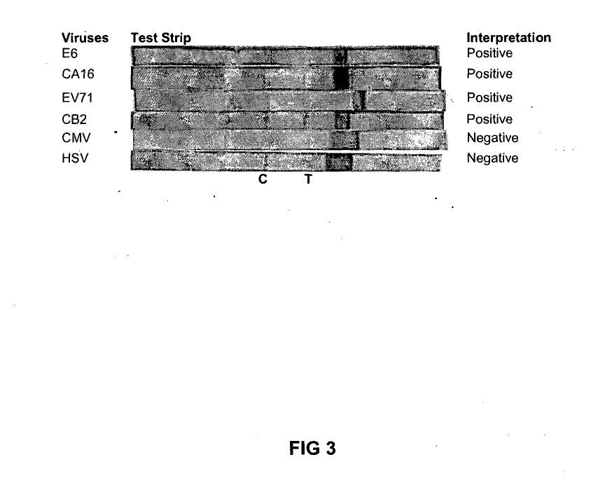

FIG. 3 shows the experimental results of the test runs using an embodiment of a lateral flow immunochromatographic assay and lateral flow test strip of the present disclosure to detect different types of viruses, wherein C is the control line and T is the test line. As shown in FIG. 3, the lateral flow immunochromatographic assay and lateral flow test strip are specific for enteroviruses. For example, each of the test strips used to test saliva fluid samples containing the enteroviruses E6, CA16, EV71 and CB2 showed the formation of a red colour line thus providing a positive test and confirming that each of the saliva fluid samples contained an enterovirus. However, each of the test strips used to test saliva fluid samples containing the non-enteroviruses CMV and HSV did not show the formation of a red colour line thus providing a negative test and confirming that the saliva fluid samples did not contain an enterovirus.

Each of test strips provided results in 10 minutes from the time of adding the sample fluid saliva to the sample pad.

As demonstrated by the experimental results, the point-of-care lateral flow immunochromatographic assay and lateral flow test strip of the present disclosure exhibit an improved efficiency in detecting enteroviruses.

While various aspects and embodiments have been disclosed herein, it will be apparent that various other modifications and adaptations of the invention will be apparent to the person skilled in the art after reading the foregoing disclosure without departing from the spirit and scope of the invention and it is intended that all such modifications and adaptations come within the scope of the appended claims. The various aspects and embodiments disclosed herein are for purposes of illustration and are not intended to be limiting, with the true scope and spirit of the invention being indicated by the appended claims.

1. A lateral flow test strip for detecting the presence of an enterovirus, wherein the test strip comprises:
a conjugate pad comprising a plurality of detector molecules; and
a test line comprising a plurality of capture molecules;
wherein the test strip detects an enterovirus.

2. The lateral flow test strip of claim 1, wherein the test strip is specific for detecting enteroviruses.

3. The lateral flow test strip of claim 1, wherein the test strip is 100% specific for detecting enteroviruses.

4. The lateral flow test strip of claim 1, wherein each of the detector molecules comprises a first antigen-specific antibody conjugated to a detectable particle.

5. The lateral flow test strip of claim 4, wherein the detectable particle comprises a gold nanoparticle.

6. The lateral flow test strip of claim 4, wherein the first antigen-specific antibody is specific for a first region of a viral surface protein.

7. The lateral flow test strip of claim 1, wherein each of the capture molecules comprises a second antigen-specific antibody.

8. The lateral flow test strip of claim 7, wherein the second antigen-specific antibody is specific for a second region of the viral surface protein.

9. The lateral flow test strip of claim 1, wherein the test strip provides test results in about 30 minutes or less.

10. The lateral flow test strip of claim 1, wherein the test strip provides test results in about 20 minutes or less.

11. The lateral flow test strip of claim 1, wherein the test strip provides test results in about 10 minutes.

12. The lateral flow test strip of claim 1, wherein the test strip provides test results with an accuracy of about 90% or greater.

13. The lateral flow test strip of claim 1, wherein the test strip provides test results with an accuracy of about 95% or greater.

14. The lateral flow test strip of claim 1, wherein the test strip provides test results with an accuracy of about 99% or greater.

15. The lateral flow test strip of claim 1, wherein the test strip provides test results with an accuracy of about 100%.

16. The lateral flow test strip of claim 1, wherein the enterovirus comprises one or more enteroviruses.

17. The lateral flow test strip of claim 1, wherein the test strip is a point-of-care lateral flow test strip.

18. A method of manufacturing the lateral flow test strip of claim 1, wherein the method comprises:
making a conjugate pad;
preparing a detector molecule solution comprising detector molecules; and
soaking the conjugate pad in the detector molecule solution.

19. A diagnostic kit for detecting one or more enteroviruses, wherein the diagnostic kit comprises the lateral flow test strip of claim 1.

20. The diagnostic kit of claim 19, further comprising: a sample collection device for collecting a biological fluid sample.

21. A method of determining the presence of an enterovirus using a lateral flow immunochromatographic assay, the method comprising:
providing a sample;
providing a lateral flow test strip comprising a plurality of detector molecules and a plurality of capture molecules;
adding the sample to a sample pad of the lateral flow test strip; and
exposing the sample to the plurality of detector molecules.

22. The method of claim 21, wherein each of the detector molecules comprises a first antigen-specific antibody conjugated to a detectable particle.

23. The method of claim 22, wherein the detectable particle comprises a gold nanoparticle.

24. The method of claim 22, wherein the first antigen-specific antibody is specific for a viral antigen comprising a viral surface protein, wherein if the sample comprises an enterovirus the first antigen-specific antibody binds to the viral surface protein to form one or more first complexes, wherein if the sample does not comprise an enterovirus the first antigen-specific antibody does not bind to the viral surface protein.

25. The method of claim 24, wherein each of the capture molecules comprises a second antigen-specific antibody, wherein the one or more first complexes are exposed to the second antigen-specific antibody, wherein the second antigen-specific antibody is specific for the viral surface protein, wherein the second antigen-specific antibody binds to the viral surface protein to form one or more second complexes.

26. The method of claim 25, wherein the formation of the one or more second complexes results in the formation of a red colour line, wherein the red colour line indicates the presence of an enterovirus.

27. The method of claim 21, wherein the sample comprises a biological fluid.

28. The method of claim 21, wherein the biological fluid comprises saliva.

29. The method of claim 21, wherein the enterovirus comprises one or more enteroviruses.

30. The method of claim 21, wherein the lateral flow immunochromatographic assay is a point-of-care lateral flow immunochromatographic assay.

31. The method of claim 21, wherein the method provides test results in about 30 minutes or less.

32. The method of claim 21, wherein the method provides test results in about 20 minutes or less.

33. The method of claim 21, wherein the method provides test results in about 10 minutes.

34. The method of claim 21, wherein the method provides test results with an accuracy of about 90% or greater.

35. The method of claim 21, wherein the method provides test results with an accuracy of about 95% or greater.

36. The method of claim 21, wherein the method provides test results with an accuracy of about 99% or greater.

37. The method of claim 21, wherein the method provides test results with an accuracy of about 100% greater.
